(19) United States
(12) Patent Application Publication
(10) Pub. No.: US 2023/0018967 A1
(43) Pub. Date: Jan. 19, 2023

(54) CATHETER AND CATHETER SYSTEM

(71) Applicant: TERUMO KABUSHIKI KAISHA, Tokyo (JP)

(72) Inventors: Yu OSAWA, Kanagawa (JP); Satoshi SAWADA, Kanagawa (JP)

(21) Appl. No.: 17/953,562

(22) Filed: Sep. 27, 2022

Related U.S. Application Data
(63) Continuation of application No. PCT/JP2021/001477, filed on Jan. 18, 2021.

(30) Foreign Application Priority Data
Mar. 27, 2020 (JP) 2020-058237

(51) Int. Cl. A61M 1/30; A61M 1/36; A61M 39/24

(52) U.S. Cl. CPC A61M 1/305; A61M 1/369; A61M 39/24; A61M 2205/3368; A61M 2205/3344; A61M 2210/0693; A61M 2202/0464; A61M 2202/0208

(57) ABSTRACT

A catheter includes a tubular body to be inserted into a living body. The tubular body has a lumen that is held in fluid communication with an injection port defined in a distal end of the tubular body and a suction port near a proximal end of the tubular body, and the suction port is closed when the injection port is open, and the injection port is closed when the suction port is open.

### CROSS REFERENCE TO RELATED APPLICATIONS

The present disclosure is a continuation of and claims benefit to PCT/JP2021/001477 filed on Jan. 18, 2021, entitled “CATHETER AND CATHETER SYSTEM” which claims priority to Japanese Patent Application No. 2020-058237 filed on Mar. 27, 2020. The entire disclosure of the applications listed above are hereby incorporated herein by reference, in their entirety, for all that they teach and for all purposes.

### BACKGROUND

The present disclosure relates to a catheter for being inserted through the spinal cord of a patient to deliver a high oxygen solution to the brain and a catheter system incorporating such a catheter.

For treating cerebral infarctions, there have been established various treating processes including thrombolytic therapy, endovascular clot retrieval therapy, etc. However, there are quite a lot of cases where these treating processes are hardly applicable because of bleeding risk and inaccessibility to the lesion. Consequently, most treatments for cerebral infarctions have been limited to medical, not surgical, therapy.

One possible treatment for cerebral infarctions would involve giving a high oxygen solution access to the brain to deliver oxygen to the brain via a route other than blood vessels. This treatment would require a catheter to be inserted through the spinal cord to bring its distal end to a region near the brain. A catheter for being inserted into the spinal cord is disclosed, for example, in Japanese Patent Application No. JP 2016-172087A entitled “Cerebrospinal Fluid Purification System.”

### SUMMARY

The catheter disclosed in Japanese Patent Application No. JP 2016-172087A is a device to be inserted into the spinal cord to remove the cerebrospinal fluid therefrom and, after the cerebrospinal fluid has been processed in some way, return it to the spinal cord. Therefore, the disclosed catheter cannot be used for the treatment described above. A tubular body having a plurality of lumens therein is required to introduce and draw a cerebrospinal fluid into and from a living organism. However, since a spinal canal, which is the hollow part of a spinal cord, is very thin, each of the lumens is required to be thin too. Consequently, the catheter may possibly fail to keep the fluid sufficiently flowable in the lumens.

In order to solve the problem described above, it is therefore an object of the present disclosure to provide a catheter and a catheter system that are capable of introducing and drawing a fluid into and from a living body through a single lumen.

In order to achieve the above object, there is provided in accordance with an aspect of the present disclosure a catheter including a tubular body to be inserted into a living body. The tubular body has a lumen therein (e.g., the lumen is disposed in an interior of the tubular body) that is held in fluid communication with an injection port defined in a distal end of the tubular body and a suction port near a proximal end thereof, and the suction port is closed when the injection port is open, and the injection port is closed when the suction port is open.

In order to achieve the above object, there is provided in accordance with another aspect of the present disclosure a catheter system including the catheter described above, an injection actuator for supplying a fluid to the injection port through the lumen in the tubular body, a suction actuator for drawing in a fluid from the suction port through the lumen in the tubular body, a detector for detecting an intracranial pressure of the living body, and a controller for controlling the injection actuator and the suction actuator. The controller alternately controls the injection actuator and the suction actuator in order to keep the intracranial pressure detected by the detector within a certain range.

With the catheter arranged as described above, since either the injection port or the suction port with which the lumen is held in fluid communication is open at a time, the catheter is able to introduce a cerebrospinal fluid into the living body and draw a cerebrospinal fluid from the living body through the single lumen.

The tubular body may have a one-way valve disposed therein that allows a fluid to flow in a direction from out of the lumen, and the suction port may have an opening capable of being selectively opened and closed. Therefore, either the injection port or the suction port can be open at a time.

The tubular body may include a first tube having the injection port and the suction port and a second tube rotatable or longitudinally slidable with respect to the first tube. The second tube may be disposed in the first tube, or the first tube may be disposed in the second tube. The second tube may have a fluid communication port that is brought selectively into and out of fluid communication with the suction port when the second tube is rotated or longitudinally slid with respect to the first tube. The suction port can thus be selectively opened and closed by a simple manipulation of the tubular body.

The second tube may have the one-way valve in a distal end portion thereof. The one-way valve may be disposed as a protrusive member on the distal end of the second tube, though it remains positioned in the lumen when the second tube is inserted in the first tube.

The suction port may be in the form of a plurality of ports. As the plurality of suction ports allow the catheter to draw in a cerebrospinal fluid efficiently, the catheter is able to minimize an increase in the intracranial pressure.

The suction port may have a one-way valve that allows a fluid to flow in a direction from outside of the tubular body into the lumen. Consequently, the catheter is capable of introducing and drawing a cerebrospinal fluid through the single tubular body.

The catheter system thus configured is capable of continuously introducing and drawing in a cerebrospinal fluid while maintaining the intracranial pressure within a certain range.

The detector may measure the pressure of the cerebrospinal fluid. The detector may thus detect the state of the cerebrospinal fluid.

The detector may measure the oxygen concentration of the cerebrospinal fluid. The detector may thus detect the state in the cranium.

The controller may have a temperature controller for controlling the temperature of the fluid supplied to the injection port. The temperature of the fluid introduced into the living body can thus be set to a desired value for an enhanced treatment effect.

The catheter system may further include a sensor for measuring the oxygen concentration of the fluid supplied to the injection port and the oxygen concentration of the fluid drawn in from the suction port, and the controller may calculate an oxygen consumption rate on the basis of values measured by the sensor. Consequently, the controller can monitor the oxygen consumption rate of the brain tissue.

### BRIEF DESCRIPTION OF THE DRAWINGS

FIG. 1 is a front elevational view of a catheter according to an embodiment of the present disclosure;

FIG. 2A is a front elevational view of the catheter according to the embodiment of the present disclosure, illustrating the manner in which a plurality of suction ports and a plurality of fluid communication ports are held in fluid communication with each other;

FIG. 2B is a front elevational view of the catheter according to the embodiment of the present disclosure, illustrating the manner in which the suction ports and the fluid communication ports are held out of fluid communication with each other;

FIG. 3A shows an enlarged fragmentary cross-sectional view of a distal end portion of the catheter, illustrating the manner in which a one-way valve in the distal end portion operates in a first state according to embodiments of the present disclosure;

FIG. 3B shows an enlarged fragmentary cross-sectional view of the distal end portion of the catheter, illustrating the manner in which the one-way valve in the distal end portion operates in a second state according to embodiments of the present disclosure;

FIG. 3C shows an enlarged fragmentary cross-sectional view of the distal end portion of the catheter, illustrating the manner in which the one-way valve in the distal end portion operates in a third state according to embodiments of the present disclosure;

FIG. 4 is a block diagram of a catheter system that incorporates the catheter according to the embodiment of the present disclosure;

FIG. 5A is a front elevational view of a catheter according to a first modification of the embodiment of the present disclosure, illustrating the manner in which a plurality of suction ports and a plurality of fluid communication ports are held in fluid communication with each other;

FIG. 5B is a front elevational view of the catheter according to the first modification of the embodiment of the present disclosure, illustrating the manner in which the suction ports and the fluid communication ports are held out of fluid communication with each other;

FIG. 6 is a front elevational view of a catheter according to a second modification of the embodiment of the present disclosure, with a first tubular body and a second tubular body being separate from each other;

FIG. 7 is a front elevational view of the catheter according to the second modification of the embodiment of the present disclosure, with the second tubular body inserted in the first tubular body; and

FIG. 8 is a front elevational view of a catheter according to a third modification of the embodiment of the present disclosure.

### DETAILED DESCRIPTION

Embodiments of the present disclosure will be described in detail below with reference to the accompanying drawings. In the drawings, dimensional ratios may be depicted exaggerated for illustrative purposes and may be different from actual dimensional ratios. In the description that follows, a catheter according to an embodiment of the present disclosure has opposite ends or end portions, and one of the ends or end portions that leads when the catheter is inserted into a living organism will be referred to as a “distal end” or a “distal end portion” whereas the other end or end portion that trails and is operated by the user will be referred to as a “proximal end” or a “proximal end portion.” The catheter includes various components such as a first tube and a second tube, and each of these components has opposite ends or end portions that will be similarly referred to.

When the catheter according to the present embodiment is in use, it is inserted into the spinal canal of a patient inflicted by a cerebral infarction from the lumbar spine until the distal end of the catheter is brought to a region near the brain. When the distal end of the catheter has reached the region near the brain, the catheter introduces a high oxygen solution as an artificial cerebrospinal fluid into the brain and draws the cerebrospinal fluid from the brain under suction to the proximal end portion of the catheter, thereby treating the cerebral infarction.

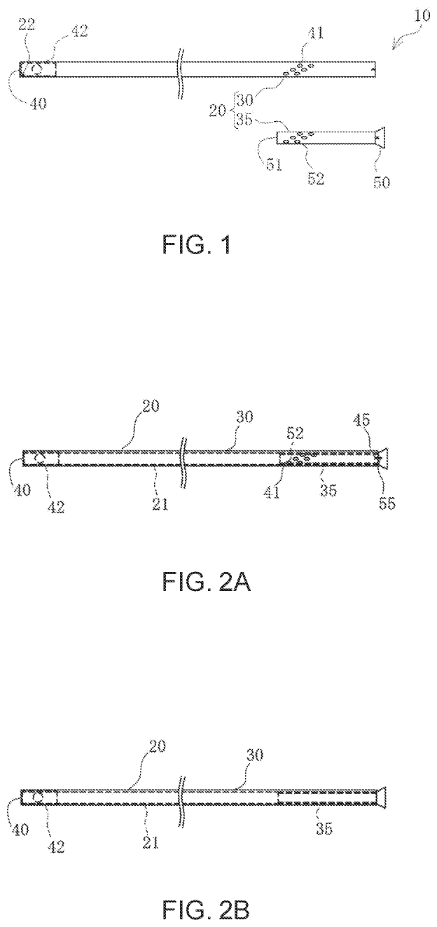

As illustrated in FIG. 1, the catheter 10, according to at least one embodiment of the present disclosure has an elongate tubular body or assembly 20. The tubular body 20 includes a first tube 30 and a second tube 35. When the catheter 10 is in use, the second tube 35 is inserted in the first tube 30. The first tube 30 has an injection port 40 that is defined in the distal end thereof and that is open forwardly away from the proximal end of the first tube 30. The first tube 30 also has a plurality of suction ports 41 defined in a side wall thereof at a position remote from the distal end thereof. The second tube 35 may have any desired length and, when inserted in the first tube 30, may extend to have its distal end near the distal end of the first tube 30. However, the second tube 35 may be arranged shorter in length than that of the first tube 30, as illustrated in FIG. 1. According to embodiments of the present disclosure, the second tube 35 is inserted in the first tube 30 as illustrated. However, in some embodiments, the first tube 30 may be inserted in the second tube 35.

In some embodiments, the suction ports 41 include six suction ports 41. The plurality of suction ports 41 allow the catheter 10 in the living organism to draw the cerebrospinal fluid efficiently under suction. However, the number of suction ports 41 is not limited to six. The positions of the suction ports 41 are not limited to the positions illustrated in FIG. 1 either, but may be located at a position closer to the distal end of the first tube 30. However, the suction ports 41 are positioned closer to the proximal end of the first tube 30 than the distal end of the second tube 35 inserted in the first tube 30. The suction ports 41 should preferably be positioned in the proximal end portion of the first tube 30.

A one-way valve 42 is disposed in the distal end portion of the first tube 30. The one-way valve 42 allows a fluid to flow in a direction from the proximal end portion of the first tube 30 through the first tube 30 toward the injection port 40 and prevents a fluid from flowing in an opposite direction from the injection port 40 through the first tube 30 toward the proximal end portion thereof.

A detector 22 for detecting the intracranial pressure of the patient is disposed in the distal end portion of the first tube 30. According to at least one embodiment of the present disclosure, the detector 22 includes an intracranial pressure sensor.

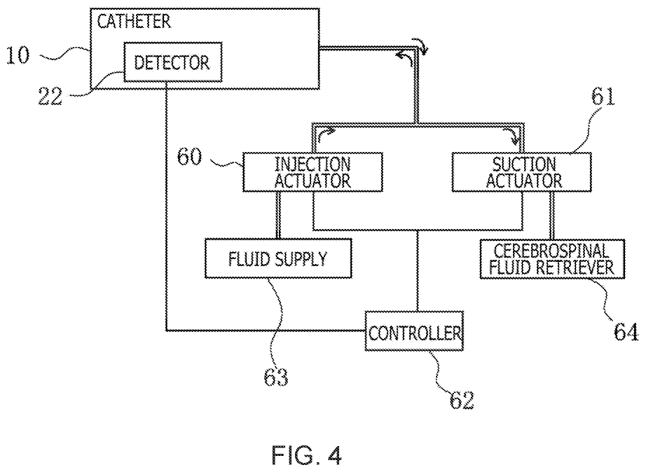

The second tube 35 has an opening 51 that is defined in the distal end thereof and that is open forwardly away from the proximal end of the second tube 35. The second tube 35 also has a plurality of fluid communication holes 52 defined in a side wall thereof at a position close to the distal end thereof. The second tube 35 that is inserted in the first tube 30 is circumferentially movable with respect to the first tube 30 to bring the fluid communication holes 52 selectively into and out of fluid communication with the suction ports 41 of the first tube 30. The second tube 35 further has a funnel-shaped inlet 50 on the proximal end thereof. The funnel-shaped inlet 50 will be connected to a fluid supply 63 (see FIG. 4), to be described later, that supplies an artificial cerebrospinal fluid with a high oxygen content and a cerebrospinal fluid retriever 64 (see FIG. 4), to be described later, that retrieves a cerebrospinal fluid.

The tubular body 20, that includes both the first tube 30 and the second tube 35, should preferably be made of a material that is pliable or flexible to a certain extent; non-limiting examples include polyolefin such as polyethylene, polypropylene, polybutene, ethylene-propylene copolymer, ethylene-vinyl acetate copolymer, ionomer, or a mixture of two or more of these compounds, fluororesin such as soft polyvinyl chloride resin, polyamide, polyamide elastomer, polyester, polyester elastomer, or polyurethane, polytetrafluoroethylene, engineering plastic such as polyether ether ketone (PEEK), silicone rubber, latex rubber, or the like.

As illustrated in FIG. 2A, the tubular body 20 is completed when the second tube 35 is inserted into the first tube 30 through the proximal end thereof and integrally combined with the first tube 30. The tubular body 20 has a single lumen 21 defined therein that extends all the way from the proximal end to the distal end thereof. The gap between the first tube 30 and the second tube 35 should desirably be sealed by a fluid-tight seal such as a packing.

The tubular body 20 can provide fluid communication between the lumen 21 and the exterior of the tubular body 20 when the suction ports 41 of the first tube 30 and the fluid communication holes 52 of the second tube 35 are circumferentially aligned with each other or, in other words, in the same circumferential position. When a negative pressure is developed in the lumen 21 while the suction ports 41 and the fluid communication holes 52 are being circumferentially aligned with each other, a fluid is drawn from the exterior of the tubular body 20 through the suction ports 41 and the fluid communication holes 52 into the lumen 21. At the same time, the negative pressure developed in the lumen 21 causes the one-way valve 42 to prevent a fluid from flowing in from the injection port 40. In other words, at this time, the suction ports 41 are open and the injection port 40 is closed.

As illustrated in FIG. 2B, the first tube 30 and the second tube 35 can be circumferentially moved with respect to each other to bring the suction ports 41 of the first tube 30 and the fluid communication holes 52 of the second tube 35 out of fluid communication with each other. When the suction ports 41 and the fluid communication holes 52 are brought out of fluid communication with each other, the suction ports 41 are closed. When a positive pressure is developed in the lumen 21 at this time, a fluid is discharged from the lumen 21 through the injection port 40 and injected into a living tissue ahead of the injection port 40. The positive pressure developed in the lumen 21 causes the one-way valve 42 to allow the fluid to flow in the lumen 21 toward the injection port 40. At this time, the injection port 40 is open and the suction ports 41 are closed.

The first tube 30 and the second tube 35 have respective markers 45 and 55 on the respective proximal ends thereof. The markers 45 and 55 are circumferentially aligned with each other when the suction ports 41 and the fluid communication holes 52 are circumferentially aligned with each other, i.e., in the same circumferential position. Consequently, when the markers 45 and 55 are circumferentially aligned with each other, the user knows that the suction ports 41 and the fluid communication holes 52 are in the same circumferential position. The first tube 30 and the second tube 35 can manually be moved in circumferential directions with respect to each other. However, the first tube 30 and the second tube 35 may be coupled to a rotary actuator, not depicted, such that they can automatically be moved circumferentially with respect to each other.

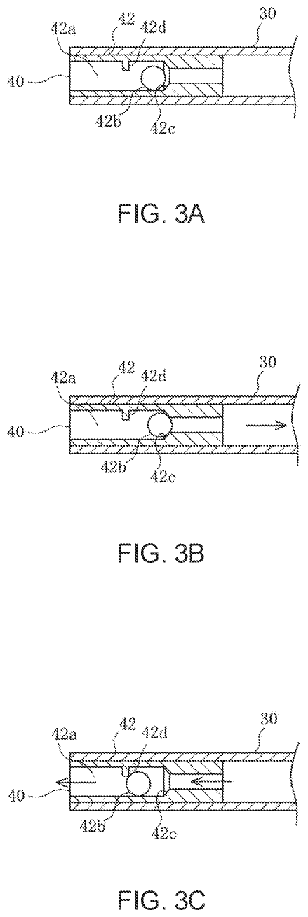

As illustrated in FIG. 3A, the one-way valve 42 has a fluid passage 42a extending all the way from the proximal end to distal end thereof. The fluid passage 42a is defined in a tubular member fitted in the first tube 30. The tubular member has a stepped fully circumferential abutment surface 42c extending fully circumferentially and protruding radially inwardly from a circumferential portion of the tubular member and a stepped partial abutment ledge 42d extending partially circumferentially and protruding radially inwardly from a circumferential portion of the tubular member. The stepped fully circumferential abutment surface 42c is axially spaced from the stepped partial abutment ledge 42d and is positioned more closely to the proximal end of the tubular member than the stepped partial abutment ledge 42d. The one-way valve 42 also includes a spherical valve element or ball 42b movably disposed between the stepped fully circumferential abutment surface 42c and the stepped partial abutment ledge 42d.

As illustrated in FIG. 3B, when the lumen 21 is evacuated to develop a negative pressure in the lumen 21, the ball 42b moves into abutment against the stepped fully circumferential abutment surface 42c (e.g., held in place against the abutment surface 42 by the negative pressure, or suction). Since the surface of the ball 42b is fully circumferentially held in abutment against the stepped fully circumferential abutment surface 42c, it closes the fluid passage 42a of the one-way valve 42. Therefore, no fluid flows in the lumen 21 from the distal end to the proximal end of the tubular body 20 when the ball 42b is in the state illustrated in FIG. 3B.

As illustrated in FIG. 3C, when the lumen 21 is pressurized to develop a positive pressure in the lumen 21, the ball 42b is pushed into abutment against the stepped partial abutment ledge 42d and/or away from contacting the circumferential abutment surface 42c. Since the stepped partial abutment ledge 42d extends only partially circumferentially, a clearance is created between the ball 42b and a wall surface of the tubular member that defines the fluid passage 42a. Therefore, the fluid passage 42a remains open, allowing a fluid to flow in the lumen 21 from the proximal end to the distal end of the tubular body 20 when the ball 42b is in the state illustrated in FIG. 3C. Though the stepped partial abutment ledge 42d extends partially circumferentially in the illustrated embodiment, it may extend fully circumferentially. The distance from the stepped partial abutment ledge 42d to the stepped fully circumferential abutment surface 42c should be larger than the diameter of the ball 42b disposed in the fluid passage 42a. The stepped fully circumferential abutment surface 42c may be provided by the distal end of the second tube 35. In a case where the stepped fully circumferential abutment surface 42c is provided by the distal end of the second tube 35, the inside diameter of the stepped fully circumferential abutment surface 42c needs to be smaller than the diameter of the ball 42b.

A catheter system that includes the catheter 10 according to the present embodiment will be described below with reference to the schematic diagram of FIG. 4. In FIG. 4, the single lines interconnecting some components of the catheter system indicate that they are electrically interconnected, whereas the double lines interconnecting some components of the catheter system indicate that they are interconnected by fluid passages. The catheter system includes the catheter 10, an injection actuator 60 for delivering an artificial cerebrospinal fluid from the fluid supply 63 to the catheter 10, a suction actuator 61 for drawing a cerebrospinal fluid from the patient through the catheter 10 into the cerebrospinal fluid retriever 64, and a controller 62 for controlling the injection actuator 60 and the suction actuator 61. The controller 62 alternately controls the injection actuator 60 and the suction actuator 61 in order to keep the intracranial pressure detected by the detector 22 within a certain range.

A treatment process using the catheter 10 according to the present embodiment will be described below. In the treatment process, the catheter 10 that has been connected to injection actuator 60, the suction actuator 61, and the controller 62 is percutaneously inserted into a living body, i.e., the body of a patient, and introduced into the spinal canal through a space between two lumbar vertebrae or a lumbar vertebra and the sacrum. Specifically, the catheter 10 is introduced into the spinal canal through the space between the lumbar vertebrae L3 and L4, or the space between the lumbar vertebrae L4 and L5, or the space between the lumbar vertebra L5 and the sacral vertebra S1. However, the catheter 10 may be introduced into the spinal canal from a position other than these spaces.

The catheter 10 introduced into the spinal canal is pushed toward the brain until the injection port 40 reaches a position near the brain. It is desirable that the catheter 10 be inserted up to the cisterna magna.

When the catheter 10 has been inserted in position, the intracranial pressure that represents the pressure in the spinal canal is detected by the detector 22. The intracranial pressure should desirably be in the range from 60 to 180 mmH2O, and should not exceed 180 mmH2O. Then, the first tube 30 and the second tube 35 are rotated relatively to each other to bring the suction ports 41 and the fluid communication holes 52 out of fluid communication with each other. Thereafter, the controller 62 controls the injection actuator 60 to supply an artificial cerebrospinal fluid with a high oxygen content from the fluid supply 63 to the catheter 10. Under a positive pressure developed in the lumen 21 of the catheter 10, the supplied artificial cerebrospinal fluid is discharged through the one-way valve 42 from the injection port 40 and delivered to the brain. At this time, the injection port 40 is open and the suction ports 41 are closed.

While the artificial cerebrospinal fluid is being delivered to the brain, the detector 22 detects intracranial pressure from time to time. When a predetermined amount of artificial cerebrospinal fluid has been introduced into the spinal canal or if the intracranial pressure has risen beyond the above range due to the supplied artificial cerebrospinal fluid, the controller 62 stops operating the injection actuator 60. Then, in order to draw a cerebrospinal fluid from the spinal canal, the first tube 30 and the second tube 35 are rotated relatively to each other to bring the suction ports 41 and the fluid communication holes 52 into fluid communication with each other. Thereafter, the controller 62 controls the suction actuator 61 to develop a negative pressure in the lumen 21 of the catheter 10. The cerebrospinal fluid is then drawn from the spinal canal through the fluid communication holes 52 and the suction ports 41 into the lumen 21 and retrieved through the funnel-shaped inlet 50 into the cerebrospinal fluid retriever 64. At this time, the suction ports 41 are open and the injection port 40 is closed.

The catheter system may have a safety mechanism for stopping the cerebrospinal fluid from flowing into the spinal canal when the intracranial pressure measured by an intracranial pressure sensor reaches a preset threshold value for the intracranial pressure. Moreover, the catheter system may have a sensor for measuring the oxygen saturation level of the artificial cerebrospinal fluid introduced into the brain and the oxygen saturation level of the cerebrospinal fluid drawn from the spinal canal. The controller 62 may then be able to calculate an oxygen consumption rate of the brain tissue from the measured oxygen saturation levels, so that the controller 62 can monitor the oxygen consumption rate.

The cerebrospinal fluid that has been retrieved by the cerebrospinal fluid retriever 64 is discarded as it is. Alternatively, the retrieved cerebrospinal fluid may be filtered to remove unwanted components therefrom, and then sent back to the fluid supply 63 or the injection actuator 60 and supplied together with the artificial cerebrospinal fluid to the brain.

When a predetermined amount of cerebrospinal fluid has been drawn in from the spinal canal or if the intracranial pressure has risen beyond the above range due to the drawn cerebrospinal fluid, the controller 62 stops operating the suction actuator 61. Thereafter, the introduction of an artificial cerebrospinal fluid and the drawing-in of a cerebrospinal fluid are repeated similarly.

When the introduction of an artificial cerebrospinal fluid and the drawing-in of a cerebrospinal fluid have been repeated a certain number of times or over a certain period of time, the catheter 10 is pulled out of the patient, and the treatment process is ended. The treatment process may be ended on the condition that the removal of any blood clots from the patient has been confirmed.

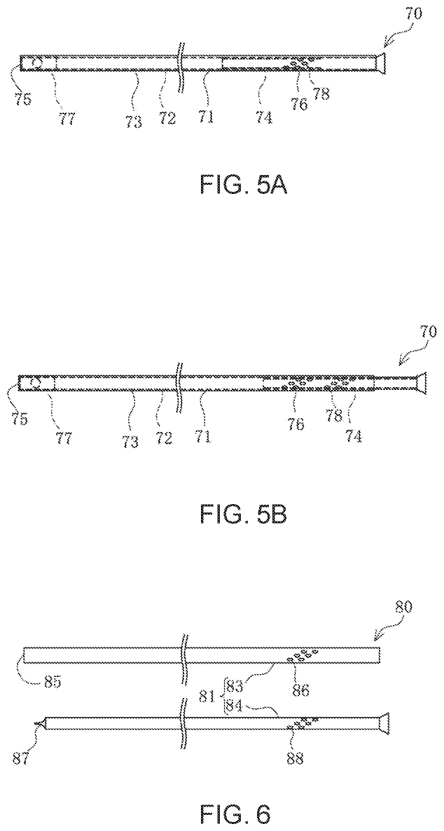

A first modification of the embodiment will be described below with reference to FIGS. 5A and 5B. As illustrated in FIG. 5A, a catheter 70 according to the first modification has an elongate tubular body or assembly 71 including a first tube 73 and a second tube 74. The tubular body 71 has a lumen 72 defined therein. The first tube 73 has an injection port 75 defined in a distal end thereof and a plurality of suction ports 76 defined in a side wall thereof. The catheter 70 includes a one-way valve 77 disposed in the lumen 72 near the distal end thereof. The second tube 74 has a plurality of fluid communication holes 78 defined in a side wall thereof for fluid communication with the suction ports 76.

The second tube 74 is slidably inserted in the first tube 73 and is longitudinally or axially slidable with respect to the first tube 73. As illustrated in FIG. 5B, when the second tube 74 is axially slid toward the proximal end of the first tube 73, the suction ports 76 and the fluid communication holes 78 can be brought out of fluid communication with each other. According to the first modification, therefore, the first tube 73 and the second tube 74 are axially slidable, instead of circumferentially movable, with respect to each other, to bring the suction ports 76 and the fluid communication holes 78 selectively into and out of fluid communication with each other. In some embodiments, the first tube 73 and the second tube 74 may be keyed with one another preventing rotation of the first tube 73 relative to the second tube 74, or vice versa. For example, the first tube 73 and the second tube 74 may be rotationally locked to one another about a common axis while still allowing selective movement, or translation, between the first tube 73 and the second tube 74 along the common axis.

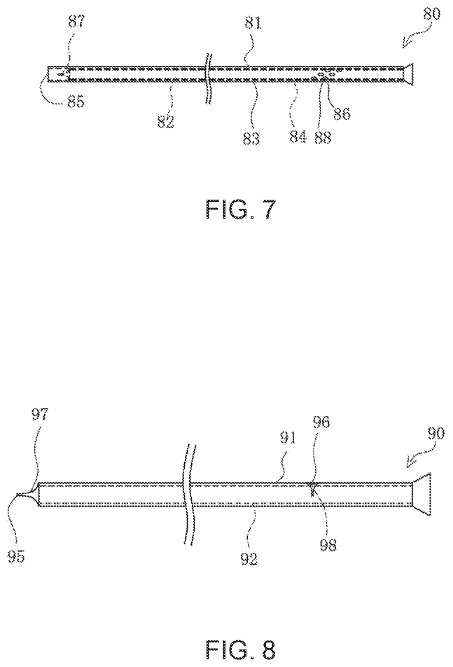

A second modification of the embodiment will be described below with reference to FIGS. 6 and 7. As illustrated in FIG. 6, a catheter 80 according to the second modification has an elongate tubular body or assembly 81 including a first tube 83 and a second tube 84. The first tube 83 has an injection port 85 defined in a distal end thereof and a plurality of suction ports 86 defined in a side wall thereof. The second tube 84 has a plurality of fluid communication holes 88 defined in a side wall thereof for fluid communication with the suction ports 86. The second tube 84 is of a length close to the length of the first tube 83 and has a one-way valve 87 disposed on the distal end thereof. The one-way valve 87 is a duckbill check valve that allows a fluid to flow in a direction from the inside toward outside of the second tube 84 and prevents a fluid from flowing in an opposite direction from the outside to inside of the second tube 84.

As illustrated in FIG. 7, when the catheter 80 is in use, the second tube 84 is inserted in the first tube 83, creating a lumen 82 in the tubular body 81 in fluid communication with the injection port 85 and the suction ports 86. The distal end of the second tube 84 inserted in the first tube 83 is disposed in the vicinity of the injection port 85 of the first tube 83. The first tube 83 and the second tube 84 inserted therein are circumferentially movable with respect to each other to bring the suction ports 86 and the fluid communication holes 88 selectively into and out of fluid communication with each other. Alternatively, the first tube 83 and the second tube 84 inserted therein may be axially movable with respect to each other to bring the suction ports 86 and the fluid communication holes 88 selectively into and out of fluid communication with each other. The one-way valve 87 that is a duckbill check valve may be disposed on the second tube 84.

A third modification of the embodiment will be described below with reference to FIG. 8. As illustrated in FIG. 8, a catheter 90 according to the third modification has an elongate tubular body 91 having a lumen 92 defined therein. The tubular body 91 has an injection port 95 defined in a distal end thereof that incorporates a one-way valve 97. The tubular body 91 also has a suction port 96 defined in a side wall thereof that incorporates a one-way valve 98. The one-way valve 97 at the injection port 95 and the one-way valve 98 at the suction port 96 are each a duckbill check valve.

The one-way valve 97 at the injection port 95 allows a fluid to flow in a direction out of the lumen 92 through the injection port 95 into the exterior of the tubular body 91 and prevents a fluid from flowing in a direction from the outside of the tubular body 91 into the lumen 92 through the injection port 95. The one-way valve 98 at the suction port 96 allows a fluid to flow in a direction from the outside of the tubular body 91 into the lumen 92 through the suction port 96 and prevents a fluid from flowing in a direction from outside of the tubular body 91 into the lumen 92 through the suction port 96. Therefore, when a positive pressure is developed in the lumen 92, the injection port 95 is open and the suction port 96 is closed, and when a negative pressure is developed in the lumen 92, the injection port 95 is closed and the suction port 96 is open. Consequently, the catheter 90 is capable of selectively switching between the introduction of an artificial cerebrospinal fluid and the drawing-in of a cerebrospinal fluid through the single tubular body 91.

As described above, the catheter 10 according to the present embodiment includes the tubular body 20 to be inserted into a living body, the tubular body 20 having the lumen 21 therein that is held in fluid communication with the injection port 40 in the proximal end of the tubular body 20 and the suction port 41 near the proximal end of the tubular body 20. The suction port 41 is closed when the injection port 40 is open, and the injection port 40 is closed when the suction port 41 is open. Since either the injection port 40 or the suction port 41 is open at a time, the catheter 10 is able to introduce a cerebrospinal fluid into the living body and draw a cerebrospinal fluid from the living body through the single lumen 21.

The tubular body 20 has the one-way valve 42 disposed therein that allows a fluid to flow in a direction out of the lumen 21. The suction port 41 may have an opening that can be selectively opened and closed. Therefore, either the injection port 40 or the suction port 41 is open at a time.

The tubular body 20 may include the first tube 30 having the injection port 40 and the suction port 41 and the second tube 35 that is disposed in the first tube 30 and that is rotatable or longitudinally slidable with respect to the first tube 30. The second tube 35 may have the fluid communication holes 52 that are brought selectively into and out of fluid communication with the suction ports 41 when the second tube 35 is rotated or longitudinally slid with respect to the first tube 30. The suction port 41 can thus be selectively opened and closed by a simple manipulation of the tubular body 20.

The second tube 84 may have the one-way valve 87 on the distal end thereof. The one-way valve 87 may be disposed as a protrusive member on the distal end of the second tube 84, though it remains positioned in the lumen 82 when the second tube 84 is inserted in the first tube 83.

The suction port 41 may be in the form of a plurality of ports. As the plurality of suction ports 41 allow the catheter 10 to draw in a cerebrospinal fluid efficiently, the catheter 10 is able to minimize an increase in the intracranial pressure.

The suction port 96 may incorporate the one-way valve 98 that allows a fluid to flow in a direction from the outside of the tubular body 91 into the lumen 92 through the injection port 40. This allows the catheter 90 to introduce and draw in an artificial cerebrospinal fluid with the single tubular body 91.

Further, the catheter system according to the present embodiment includes the catheter 10, the injection actuator 60 for supplying a fluid to the injection port 40 through the lumen 21 in the tubular body 20, the suction actuator 61 for drawing in a fluid from the suction port 41 through the lumen 21 in the tubular body 20, the detector 22 for detecting the intracranial pressure, and the controller 62 for controlling the injection actuator 60 and the suction actuator 61. The controller 62 alternately controls the injection actuator 60 and the suction actuator 61 in order to keep the intracranial pressure detected by the detector 22 within a certain range. The catheter system thus configured is capable of continuously introducing and drawing in a cerebrospinal fluid while maintaining the intracranial pressure within a certain range.

The present disclosure is not limited to the illustrated embodiment described above, and various changes and modifications may be made in the embodiment within the scope of the present disclosure. For example, though the detector 22 includes an intracranial pressure sensor in the embodiment, the intracranial pressure may be detected by other means. For example, the detector may include a sensor for detecting the flow rate of a fluid in the catheter 10 or a tube connected to the catheter 10. In this case, the controller 62 of the catheter system alternately controls the injection actuator 60 and the suction actuator 61 such that when a certain amount of artificial cerebrospinal fluid has been introduced into a living body, the same amount of cerebrospinal fluid is drawn from the living body. The catheter system is thus able to keep the amount of a cerebrospinal fluid in the spinal canal within a certain range, thereby keeping the intracranial pressure within a certain range.

The detector may include a sensor for detecting the oxygen concentration or oxygen saturation in the cerebrospinal fluid. In this case, the controller 62 of the catheter system alternately controls the injection actuator 60 and the suction actuator 61 so as to keep the oxygen concentration or oxygen saturation in the cerebrospinal fluid within a certain range. The detector may alternately include a sensor for detecting the partial pressure of oxygen or the oxygen saturation level of the cerebrospinal fluid. Further alternatively, the detector may include a sensor for measuring at least one of the concentration of carbon dioxide, pH, a mineral component (Na, K, or glucose), creatine, and a cerebrospinal fluid flow rate. The detector may calculate an oxygen consumption rate (VO2) or an oxygen supply rate (DO2) from the detected values of the above variables, and use the calculated value for feedback. Any of the above sensors may include an optical-fiber-type sensor, for example, to be incorporated in the catheter for detecting a desired value at a desired position in the catheter.

The fluid to be introduced from the injection port 40 into a living body may be other than an artificial cerebrospinal fluid. A solution with a high oxygen content may be a solution not harmful to the human body, e.g., a liquid of high gas solubility such as fluorocarbon, an emulsion thereof, or physiological saline. The solution may not have a high oxygen content.

The route from the fluid supply 63 to the injection port 40 of the catheter 10 may have a cooling or heating element for cooling or heating a fluid to be introduced into a living body. In a case where the fluid is circulated through the catheter system, the cooling or heating element may be provided in the route from the suction port 41 to the injection port 40 of the catheter 10. In this case, the controller 62 includes a temperature controller for controlling the temperature of the fluid introduced from the injection port 40 by controlling the cooling or heating element.

The distal end portion of the tubular body 20 may have a marker with an enhanced contrast imaging capability. The marker is effective to prevent the distal end portion of the tubular body 20 from being inserted beyond a predetermined position in the spinal cord, e.g., the cisterna magna.

The catheter 10 may be inserted into the spinal cord of a patient with the assistance of a guide wire. A guide wire can be inserted into the lumens 82 and 92 of the catheters 80 and 90 illustrated in FIGS. 6 and 8. To prevent the catheter from collapsing, a blade or other reinforcement may be placed in the catheter. The catheter 10 illustrated in FIG. 1 may have a guide wire lumen for inserting a guide wire therethrough. Moreover, a guide wire may be used as follows. The catheter 10 illustrated in FIG. 1 may be of such a structure that the first tube 30 will be inserted in the second tube 35 and the second tube 35 will extend to a position in the vicinity of the distal end of the first tube 30. With this structure, a guide wire is inserted into the second tube 35 to a predetermined position, and after the guide wire is removed, the first tube 30 is inserted into the second tube 35 and delivered to the predetermined position.

The catheter 10 may be used in treating lesions other than cerebral infarctions. For example, since it is considered to be effective to introduce a solution with a high oxygen content or forcibly circulate a cerebrospinal fluid in the treatment of brain diseases including cerebral hemorrhage, subarachnoid hemorrhage, hydrocephalus, and Alzheimer disease, and spinal cord ischemia, for example. The catheter 10 can be used in treating these diseases.

It should be understood by those skilled in tart that various modifications, combinations, sub-combinations and alterations may occur depending on design requirements and other factors insofar as they are within the scope of the appended claims or the equivalents thereof.

What is claimed is:

1. A catheter, comprising:
a tubular body configured to be inserted into a living body,
wherein the tubular body has a lumen within the tubular body, the lumen held in fluid communication with an injection port defined in a distal end of the tubular body and a suction port near a proximal end of the tubular body, and
wherein the suction port is closed when the injection port is open, and the injection port is closed when the suction port is open.

2. The catheter of claim 1,
wherein the tubular body has a one-way valve disposed in the tubular body that allows a fluid to flow in a direction from out of the lumen, and
wherein the suction port has an opening capable of being selectively opened and closed.

3. The catheter of claim 2,
wherein the tubular body includes a first tube having the injection port and the suction port and a second tube rotatable or longitudinally slidable with respect to the first tube,
wherein the second tube is disposed in the first tube, or the first tube is disposed in the second tube, and
wherein the second tube has a fluid communication port that is brought selectively into and out of fluid communication with the suction port when the second tube is rotated or longitudinally slid with respect to the first tube.

4. The catheter of claim 3, wherein the one-way valve is disposed in a distal end portion of the second tube.

5. The catheter of claim 2, wherein the suction port comprises a plurality of ports.

6. The catheter of claim 1, wherein the suction port has a one-way valve that allows a fluid to flow in a direction from outside of the tubular body into the lumen.

7. A catheter system, comprising:
a catheter, the catheter comprising:
a tubular body configured to be inserted into a living body; and
a lumen disposed in an interior of the tubular body, the lumen held in fluid communication with an injection port defined in a distal end of the tubular body and a suction port near a proximal end of the tubular body, wherein the suction port is closed when the injection port is open, and the injection port is closed when the suction port is open;
an injection actuator for supplying a fluid to the injection port through the lumen in the tubular body;
a suction actuator for drawing in a fluid from the suction port through the lumen in the tubular body;
a detector for detecting an intracranial pressure of the living body; and
a controller for controlling the injection actuator and the suction actuator,
wherein the controller alternately controls the injection actuator and the suction actuator in order to keep the intracranial pressure detected by the detector within a first range.

8. The catheter system of claim 7, wherein the detector measures a pressure of a cerebrospinal fluid of the living body.

9. The catheter system of claim 7, wherein the detector measures oxygen concentration of a cerebrospinal fluid of the living body.

10. The catheter system of claim 7, wherein the controller has a temperature controller for controlling a temperature of the fluid supplied to the injection port.

11. The catheter system of claim 7, further comprising:
a sensor for measuring oxygen concentration of the fluid supplied to the injection port and oxygen concentration of the fluid drawn in from the suction port,
wherein the controller calculates an oxygen consumption rate on a basis of values measured by the sensor.

12. The catheter system of claim 7, wherein the tubular body includes a one-way valve that allows a fluid to flow in a direction from out of the lumen, and wherein the suction port has an opening capable of being selectively opened and closed.

13. The catheter system of claim 12, wherein the tubular body includes a first tube with the injection port and the suction port and a second tube that is rotatable or longitudinally slidable with respect to the first tube, wherein the second tube is disposed in the first tube, and wherein the second tube has a fluid communication port that is brought selectively into and out of fluid communication with the suction port when the second tube is rotated or longitudinally slid with respect to the first tube.

14. The catheter system of claim 13, wherein the one-way valve is disposed in a distal end portion of the second tube.

15. The catheter system of claim 12, wherein the suction port comprises a plurality of ports.

16. The catheter system of claim 7, wherein the suction port has a one-way valve that allows a fluid to flow in a direction from outside of the tubular body into the lumen.

17. A catheter system, comprising:
a catheter, the catheter comprising:
a tubular body configured to be inserted into a living body; and
a lumen disposed in an interior of the tubular body, the lumen held in fluid communication with an injection port defined in a distal end of the tubular body and a suction port near a proximal end of the tubular body, wherein the suction port is closed when the injection port is open, and the injection port is closed when the suction port is open;
an injection actuator for supplying a fluid to the injection port through the lumen in the tubular body;
a suction actuator for drawing in a fluid from the suction port through the lumen in the tubular body; and
a detector for detecting an intracranial pressure of the living body.

18. The catheter system of claim 17, further comprising:
a controller that alternately controls the injection actuator and the suction actuator in order to keep the intracranial pressure detected by the detector within a first range.

19. The catheter system of claim 18, wherein the detector measures a pressure of a cerebrospinal fluid of the living body.

20. The catheter system of claim 18, wherein the detector measures oxygen concentration of a cerebrospinal fluid of the living body.
